(19) United States
(12) Patent Application Publication
(10) Pub. No.: US 2006/0133568 A1
(43) Pub. Date: Jun. 22, 2006

(54) System to provide megavoltage and kilovoltage radiation treatment

(75) Inventor: Terrence E. Moore, Pleasant Hill, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/015,053

(22) Filed: Dec. 17, 2004

(51) Int. Cl. A61N 5/10

(52) U.S. Cl. 378/65

(57) ABSTRACT

A system according to some embodiments may include an accelerator to emit megavoltage radiation towards a patient, an x-ray source to emit kilovoltage radiation towards the patient, and a concentrator to concentrate the kilovoltage radiation.

### BACKGROUND

1. Field

The embodiments described below relate generally to medical treatment, and more particularly to medical treatment using radiation.

2. Description

According to conventional radiation treatment, a beam of treatment radiation is directed toward a tumor located within a patient. The radiation beam delivers a predetermined dose of therapeutic radiation to the tumor according to an established treatment plan. The delivered radiation kills cells of the tumor by causing ionizations within the cells.

Conventional devices for delivering treatment radiation include linear accelerator-based devices and X-ray tube-based devices. Linear accelerator-based devices are used to deliver treatment radiation having radiation energies in the megavoltage range while X-ray tube-based devices are used to deliver treatment radiation having radiation energies in the kilovoltage range. Systems having increased flexibility and functionality are desired.

### SUMMARY

To address at least the foregoing, some embodiments provide a system, method, medium, apparatus, and means to deliver megavoltage treatment radiation to a patient, and to deliver kilovoltage treatment radiation to the patient. In some embodiments, the megavoltage treatment radiation and the kilovoltage treatment radiation are delivered simultaneously. Moreover, delivery of the kilovoltage treatment radiation may comprise emission of kilovoltage radiation, and concentration of the kilovoltage radiation to generate the kilovoltage treatment radiation.

According to some embodiments, provided are an accelerator to emit megavoltage radiation towards a patient, an x-ray source to emit kilovoltage radiation towards the patient, and a concentrator to concentrate the kilovoltage radiation. Further embodiments may include a first imaging device to acquire a first image based on the kilovoltage radiation, and a second imaging device to acquire a second image based on the megavoltage radiation.

The claims are not limited to the disclosed embodiments, however, as those in the art can readily adapt the teachings herein to create other embodiments and applications.

### BRIEF DESCRIPTION OF THE DRAWINGS

The construction and usage of embodiments will become readily apparent from consideration of the following specification as illustrated in the accompanying drawings, in which like reference numerals designate like parts, and wherein:

FIG. 1 is a perspective view of a radiation treatment system according to some embodiments;

FIG. 2 is a flow diagram of process steps according to some embodiments;

FIG. 3 is a perspective view of a radiation treatment system according to some embodiments;

FIG. 4 is a front view of the FIG. 3 radiation treatment system according to some embodiments;

FIG. 5 is a perspective view of a radiation treatment system according to some embodiments; and

FIG. 6 is a perspective view of a radiation treatment system according to some embodiments.

### DETAILED DESCRIPTION

The following description is provided to enable any person of ordinary skill in the art to make and use the claimed invention and sets forth the best mode contemplated by the inventors for carrying out the claimed invention. Various modifications, however, will remain readily apparent to those in the art.

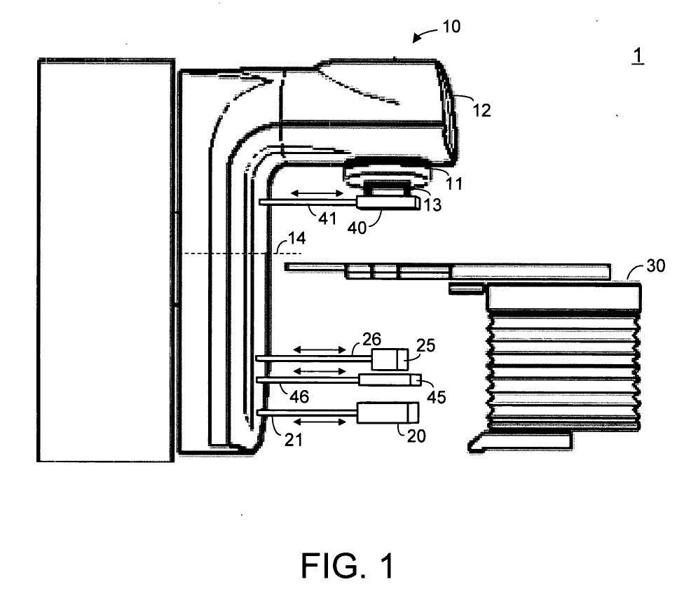

FIG. 1 illustrates radiation treatment room 1 pursuant to some embodiments. The elements of radiation treatment room 1 may be used to deliver megavoltage treatment radiation to a patient and to deliver kilovoltage treatment radiation to the patient. The elements may therefore provide more effective and/or efficient radiation treatment than otherwise available. The delivery of the two types of radiation may occur sequentially, simultaneously, or in any combination thereof. According to some embodiments, the kilovoltage treatment radiation is generated by concentrating kilovoltage radiation using concentrating systems that are or become known.

Radiation treatment room 1 includes linear accelerator 10, X-ray tube 20, and concentrator 25. Linear accelerator 10, X-ray tube 20, and concentrator 25 may be used to deliver treatment radiation according to a radiation treatment plan. More specifically linear accelerator 10 may emit megavoltage radiation towards a patient lying on table 30, and X-ray tube 20 may emit kilovoltage radiation toward the patient. Concentrator 25 may concentrate the kilovoltage radiation to generate treatment kilovoltage radiation.

Linear accelerator 10 is primarily composed of treatment head 11 and gantry 12. Treatment head 11 includes a beam-emitting device (not shown) for emitting a radiation beam used during calibration, verification, and/or treatment. The radiation beam may comprise electron, photon or any other type of megavoltage radiation. Treatment head 11 may include a target for receiving electron radiation emitted by the beam-emitting device and for generating photon radiation in response.

Beam-shaping device 13 is mounted on treatment head 11 and may receive the megavoltage radiation from treatment head 11. Device 13 may change a profile of the megavoltage radiation prior to receipt of the radiation by a patient lying on table 30. Device 13 may employ filters, collimator leaves or any other currently- or hereafter-known systems to change a shape and/or energy distribution of the megavoltage radiation emitted by treatment head 11. The resulting radiation profile may comply with a predetermined radiation treatment plan.

Treatment head 11 is fastened to a projection of gantry 12. Gantry 12 is rotatable around gantry axis 14 before, during and after radiation treatment. In some embodiments, gantry 12 may rotate clockwise and counter-clockwise around axis 14. Rotation of gantry 12 serves to rotate treatment head 11 around axis 14.

X-ray tube 20 may comprise any suitable radiation source, including but not limited to a Diabolo™ x-ray tube. In some embodiments, X-ray tube 20 emits electron, photon or any other type of radiation having energies ranging from 50 to 150 keV. The radiation emitted by X-ray tube may comprise any radiation suitable for treatment according to some embodiments.

Projection 21 couples X-ray tube 20 to gantry 12. Projection 21 may include any system or systems to move X-ray tube 20 to a desired position. According to some embodiments, X-ray tube 20 is moved toward gantry 12 prior to the emission of megavoltage radiation by linear accelerator 10. Projection 21 may then move X-ray tube 20 to a position suitable for emission of kilovoltage treatment radiation.

Concentrator 25 mat receive the kilovoltage radiation from X-ray tube 20 and generate treatment radiation based thereon. According to some embodiments, concentrator 25 includes optics such as a focusing lens for optically processing the received radiation. The focusing lens may comprise a lens for producing a convergent radiation beam from radiation emitted by X-ray tube 20. Examples of this type of lens are described in U.S. Pat. No. 6,359,963 to Cash, in U.S. Pat. No. 5,604,782 to Cash, Jr., in U.S. Patent Application Publication No. 2001/0043667 of Antonell et al., and/or elsewhere in currently or hereafter-known art.

Concentrator 25 may also include beam-shaping elements to change a profile of the kilovoltage treatment radiation prior to receipt of the radiation by a patient lying on table 30. As mentioned above, these elements may include filters, collimator leaves or any other currently- or hereafter-known systems to change a shape and/or energy distribution of kilovoltage treatment radiation.

Concentrator 25 is coupled to gantry 12 via projection 26. Projection 26 may include any system or systems to move concentrator 25 to a desired position. Concentrator 25 may be moved toward gantry 12 during a kilovoltage imaging procedure and may be moved between X-ray tube 20 and table 30 prior to kilovoltage radiation treatment.

Imaging device 40 is mounted to gantry 12 via projection 41. Imaging device 40 may acquire a projection image of a patient disposed between X-ray tube 20 and imaging device 40. Such an image may be acquired after concentrator 25 has been moved out of the path between tube 20 and imaging device 40. The image may be acquired during the delivery of the megavoltage treatment radiation. The image may be used for verification and recordation of a patient position and of an internal patient portal to which radiation is to be delivered.

Projection 41 may be configured to move imaging device 40 to the illustrated position for imaging purposes and to a second position closer to gantry 12 for the delivery of megavoltage treatment radiation. Movement to the second position may prolong the operational life of imaging device 40 by reducing its exposure to megavoltage treatment radiation. Imaging device 40 may comprise any suitable type of imaging device, including but not limited to a flat-panel imaging device using a scintillator layer and solid-state amorphous silicon photodiodes deployed in a two-dimensional array. The RID1640, offered by Perkin-Elmer®, Inc. of Fremont, Calif., is one suitable device.

Imaging device 45 may receive megavoltage treatment radiation from linac 10 to acquire a projection image of a patient disposed between treatment head 11 and imaging device 45. Such an image may be acquired at any suitable time, including during the delivery of kilovoltage treatment radiation. Imaging device 45 may comprise any suitable type of imaging device. Projection 46 may be used to move imaging device 45 to a desired position.

Embodiments are not limited to the configuration shown in FIG. 1 and described above. For example, the relative arrangement of elements 20, 25 and 45 may vary among embodiments. Moreover, the direction and degree of movement of elements 20, 25 and 45 may differ from those illustrated.

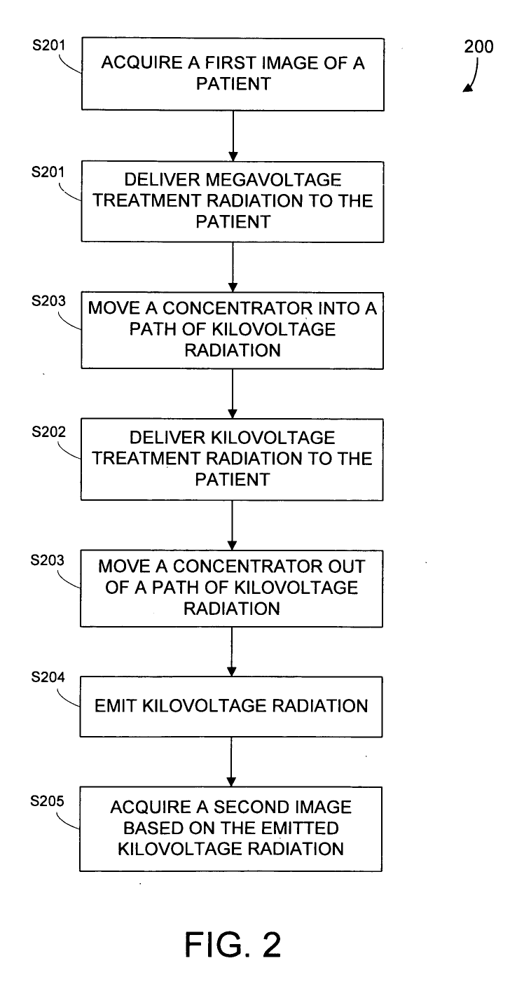

FIG. 2 is a flow diagram of process steps 200. Process steps 200 describe one of many possible processes that may be executed by a system according to some embodiments. Process steps 200 may be embodied, in whole or in part, by hardware of and/or software executed by devices including but not limited to those of FIG. 1.

Process steps 200 may be stored by any medium, including a fixed disk, a floppy disk, a CD-ROM, a DVD-ROM, a Zip™ disk, a magnetic tape, or a signal. Some or all of process steps 200 may also be stored in one or more devices. Moreover, some or all of the process steps 200 may be implemented in hardware, such as a hardware card installed in and/or discrete circuitry of linear accelerator 10.

A first image of a patient is acquired at step S201. The image may be acquired to verify the position of the patient and/or the profile of treatment radiation to be delivered. The first image may be acquired using a linear accelerator such as linear accelerator 10 and an imaging device such as imaging device 45, or using an X-ray tube and corresponding imaging device such as X-ray tube 20 and imaging device 40. In the latter case, a concentrator such as concentrator 25 may be moved out of a path between the X-ray tube and the imaging device prior to imaging. According to some embodiments, an X-ray tube and corresponding imaging device are rotated around the patient at steps S201 to acquire a three-dimensional image.

Megavoltage treatment radiation is delivered to the patient at step S202. Continuing to refer to FIG. 1 as an example, linear accelerator 10 may emit megavoltage treatment radiation at step S202. The amount, direction, shape, and/or energy of the megavoltage treatment radiation may comply with a previously-generated treatment plan. Prior to emission of the megavoltage treatment radiation, X-ray tube 20, concentrator 25 and/or imaging device 40 may be moved out of a path between treatment head 11 and imaging device 45.

Next, at step S203, a concentrator is moved into a path of kilovoltage radiation to be delivered to the patient. Step S203 may also include moving an X-ray tube into an appropriate position with respect to the patient. Kilovoltage treatment radiation is then delivered to the patient in step S204 by emitting kilovoltage radiation from the X-ray tube and concentrating the kilovoltage radiation to generate kilovoltage treatment radiation. Concentration may involve focusing the radiation as described above. In the case of the FIG. 1 apparatus, imaging device 45 may be moved out of the path between tube 20 and concentrator 25 prior to step S204. In some embodiments, megavoltage treatment radiation and kilovoltage treatment radiation are delivered simultaneously. That is, at least a portion of step S202 is performed simultaneously with at least a portion of step S204.

The concentrator is then moved out of a path of the kilovoltage radiation at step S205. Projection 26, for example, may operate at step S205 to move concentrator 25 toward gantry 12. Next, X-ray tube 20 emits kilovoltage radiation at step S206.

An image is acquired based on the emitted kilovoltage radiation at step S207. The image may be used for verification of treatment delivery and may comprise an image of the patient portal to which the kilovoltage treatment radiation was delivered at step S204. Accordingly, the kilovoltage radiation emitted at step S206 may be appropriate for acquiring such an image.

Embodiments may differ from the operation of process steps 200. For example, the kilovoltage treatment radiation may be delivered before the megavoltage treatment radiation. Images may be acquired during steps S202 and/or S204 to indicate a delivered radiation dose. In some embodiments, no images are acquired.

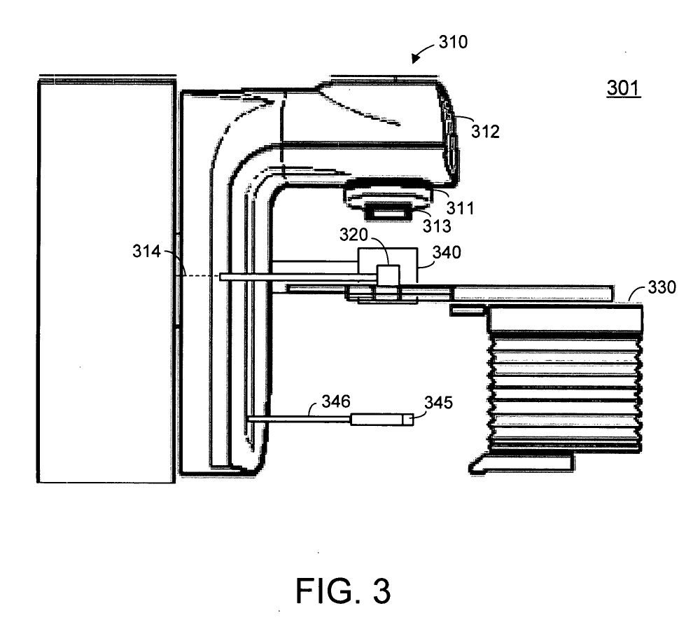

Turning now to FIG. 3, a perspective view of treatment room 301 according to some embodiments is shown. The elements of radiation treatment room 301 may be used to deliver megavoltage treatment radiation to a patient and to deliver kilovoltage treatment radiation to the patient. In some embodiments, the elements of treatment room 301 may execute process 200.

Treatment room 301 includes linear accelerator 310, X-ray tube 320, table 330, imaging device 340, and imaging device 345. Linear accelerator 310 may comprise, for example, treatment head 311, gantry 312, and beam-shaping device 313. According to some embodiments, the elements of treatment room 301 may be similar in configuration and/or functionality to the similarly-named components described in conjunction with FIG. 1. In some embodiments, fewer or more components than are shown in FIG. 3 may be included in treatment room 301.

As shown, a path between X-ray tube 320 and imaging device 340 is disposed perpendicular to a path between treatment head 311 and imaging device 345. Such an arrangement may, for example, allow the delivery of megavoltage treatment radiation without requiring movement of X-ray tube 320 or imaging device 340 out of the path of the megavoltage radiation. Similarly, the arrangement may allow kilovoltage treatment radiation to be delivered without requiring movement of treatment head 311 or imaging device 345 out of the path of the kilovoltage radiation.

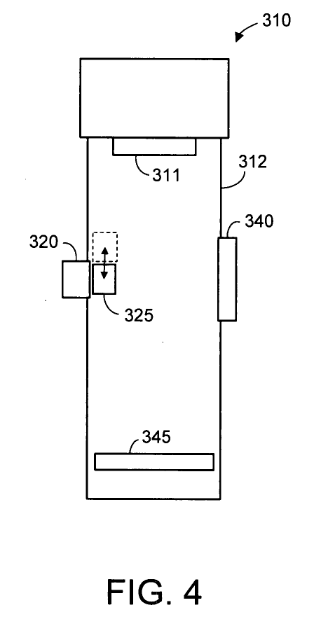

FIG. 4 shows a block diagram of a front view of some elements of treatment room 301. Concentrator 325 may receive kilovoltage radiation from X-ray tube 320 and focus the kilovoltage radiation prior to delivery thereof to a patient. Concentrator 325 may be movable out of the path of the kilovoltage radiation to a location indicated by the dotted lines of FIG. 4. Concentrator may be moved to this location prior to acquisition of an image by imaging device 323.

Treatment head 311 and X-ray tube 320 may be fixed with respect to one another. According to some embodiments, gantry 312 is rotatable around axis 314. Consequently, rotation of gantry 312 results in rotation of X-ray tube 320 and treatment head 311 around axis 314 in a fixed relationship.

According to some embodiments in which X-ray tube 320 and imaging device 340 are rotatable around a patient, X-ray tube 320 may emit imaging radiation and imaging device 340 may acquire an image based on the imaging radiation at any point during their rotation. Imaging device 340 may therefore acquire a plurality of projection images of the patient portion disposed between X-ray tube 320 and imaging device 340, with some of the images having different perspectives. These images may be used to create a three-dimensional cone beam reconstruction image of the patient portion according to currently- or hereafter-known techniques.

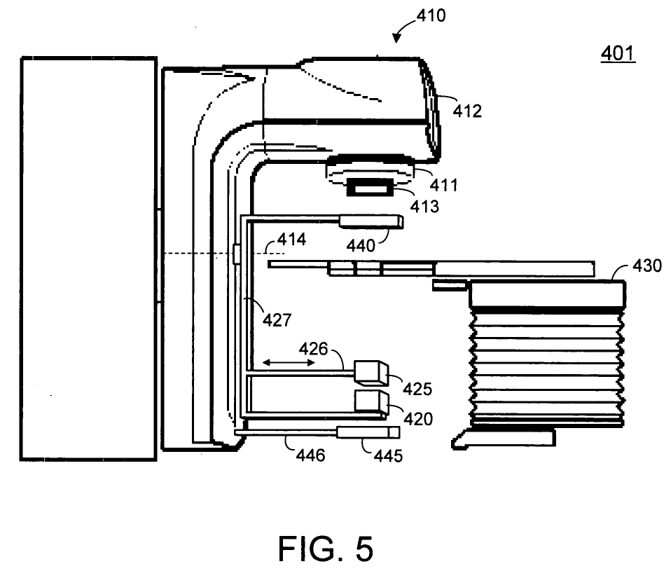

FIG. 5 comprises a perspective view of treatment room 401 according to some embodiments. The elements of radiation treatment room 401 may also be used to deliver megavoltage treatment radiation to a patient and to deliver kilovoltage treatment radiation to the patient. The elements of treatment room 401 may be employed to execute process 200.

Treatment room 401 includes linear accelerator 410, X-ray tube 420, concentrator 425, table 430, imaging device 440, and imaging device 445. Linear accelerator 410 includes treatment head 411, gantry 412, and beam-shaping device 413. The elements of treatment room 401 may be similar in configuration and/or functionality to the similarly-named components described in conjunction with FIGS. 1 and 3.

FIG. 5 shows treatment head 411, X-ray tube 420, concentrator 425, imaging device 440, and imaging device 445 disposed in line with one another. Imaging device 445 is coupled to gantry 412 via projection 446. X-ray tube 420, concentrator 425, and imaging device 440 are disposed between treatment head 411 and imaging device 445 and are each coupled to support 427. Concentrator 425 is mounted to projection 426, which may move concentrator 425 in and out of a position between X-ray tube 420 and display 440.

According to some embodiments, support 427 is rotatable to rotate X-ray tube 420, imaging device 440, and concentrator 425 around axis 414 independent from any rotation of gantry 412. Support 427 may also or alternatively be rotatable to rotate X-ray tube 420, imaging device 440, and concentrator 425 around an axis different from axis 414. In a case that support 427 is rotatable, X-ray tube 420 and imaging device 440 may be used to create a three-dimensional cone beam reconstruction image of the patient portion as described above. Concentrator 425 may be moved out of a path between X-ray tube 420 and imaging device 440 during such imaging.

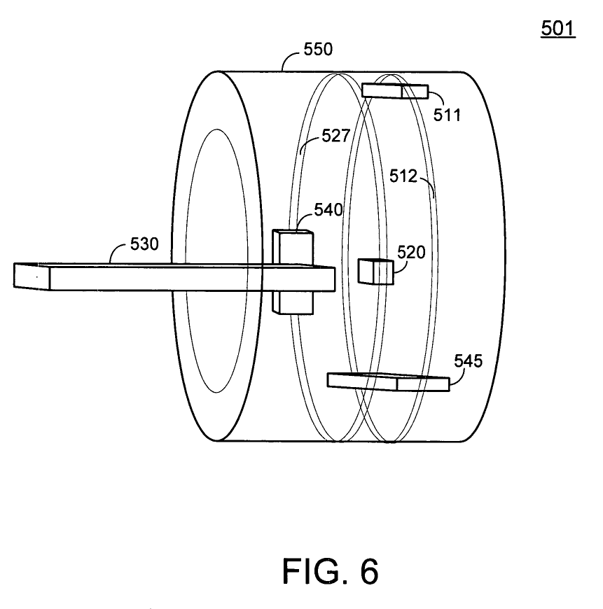

FIG. 6 illustrates a system according to further embodiments. Treatment room 501 of FIG. 6 includes treatment head 511, X-ray tube 520, imaging device 540, imaging device 545, and table 530. The elements of treatment room 501 may be similar in configuration and/or functionality to the similarly-named components described in conjunction with FIGS. 1, 3 and 5. Specifically, the elements of radiation treatment room 501 may deliver megavoltage treatment radiation and kilovoltage treatment radiation to a patient. In some embodiments, the elements of treatment room 501 may execute process 200.

Treatment head 511 and imaging device 545 are coupled to ring 512, which is in turn mounted within housing 550. Housing 550 may be similar to a computed tomography scanner housing. Also mounted within housing 550 is ring 527 to which X-ray tube 520 and imaging device 540 are also mounted. Ring 512 and ring 527 may move independently of each other to provide separate or simultaneous megavoltage radiation treatment, kilovoltage radiation treatment, megavoltage radiation imaging, and/or kilovoltage radiation imaging of a patient lying on table 530.

A concentrator (not shown) may be mounted to ring 527 or to X-ray tube 520 to receive and concentrate kilovoltage radiation emitted by X-ray tube 520 toward imaging device 540. The concentrator may be movable out of the path between X-ray tube 520 and imaging device 540 in order to provide kilovoltage radiation-based imaging of the patient. Ring 527 may therefore be rotated to acquire images for creating a three-dimensional cone beam reconstruction image of the patient.

Those in the art will appreciate that various adaptations and modifications of the above-described embodiments can be configured without departing from the scope and spirit of the claims. Therefore, it is to be understood that the claims may be practiced other than as specifically described herein.

1. An apparatus comprising:
an accelerator to emit megavoltage radiation towards a patient;
an x-ray source to emit kilovoltage radiation towards the patient;
a concentrator to receive the kilovoltage radiation and to produce a convergent kilovoltage radiation beam from the received kilovoltage radiation; and
a transport device to move the concentrator out of a path between the x-ray source and the patient.

2. An apparatus according to claim 1, further comprising:
a first imaging device to acquire a first image based on the kilovoltage radiation.

3. An apparatus according to claim 2, further comprising:
a second imaging device to acquire a second image based on the megavoltage radiation.

4. An apparatus according to claim 3, further comprising:
a first transport device to move the x-ray source and the first imaging device relative to the patient; and
a second transport device to move the linear accelerator and the second imaging device relative to the patient.

5. An apparatus according to claim 1, wherein the megavoltage radiation comprises megavoltage electron radiation, and further comprising:
a target to receive the megavoltage electron radiation and to emit megavoltage photon radiation towards the patient.

6. (canceled)

7. An apparatus according to claim 1, further comprising:
an imaging device to acquire a first image based on the kilovoltage radiation, and to acquire a second image based on the convergent kilovoltage radiation beam.

8. An apparatus according to claim 1, further comprising:
a first device to receive the megavoltage radiation and to change a profile of the megavoltage radiation prior to receipt of the megavoltage radiation by the patient; and
a second device to receive the kilovoltage radiation and to change a profile of the kilovoltage radiation prior to receipt of the kilovoltage radiation by the patient.

9. An apparatus according to claim 1, wherein the concentrator comprises at least one focusing lens.

10. A method for a system, the method comprising:
delivering megavoltage treatment radiation to a patient;
emitting kilovoltage radiation;
concentrating the kilovoltage radiation using a concentrator to produce a convergent kilovoltage radiation beam;
delivering the convergent kilovoltage radiation beam to the patient; and
moving the concentrator out of a path of the kilovoltage radiation.

11. A method according to claim 10, wherein the megavoltage treatment radiation and the convergent kilovoltage radiation beam are delivered simultaneously.

12. (canceled)

13. A method according to claim 10, wherein concentrating the kilovoltage radiation comprises:
focusing the kilovoltage radiation.

14. A method according to claim 10, further comprising:
emitting second kilovoltage radiation; and
controlling an imaging device to acquire an image based on the second kilovoltage radiation.

15. A method according to claim 10, further comprising:
acquiring an image based on the megavoltage treatment radiation.

16. A medium having processor-executable process steps stored thereon, the process steps comprising:
a step to deliver megavoltage treatment radiation to a patient;
a step to emit kilovoltage radiation;
a step to concentrate the kilovoltage radiation using a concentrator to produce a convergent kilovoltage radiation beam;
a step to deliver the convergent kilovoltage radiation beam to the patient; and
a step to move the concentrator out of a path of the kilovoltage radiation.

17. A medium according to claim 16, wherein the megavoltage treatment radiation and the convergent kilovoltage radiation beam are delivered simultaneously.

18. (canceled)

19. A medium according to claim 16, wherein the step to concentrate the kilovoltage radiation comprises:
a step to focus the kilovoltage radiation.

20. A medium according to claim 16, further comprising:
a step to emit second kilovoltage radiation; and
a step to control an imaging device to acquire and image based on the second kilovoltage radiation.

21. A medium according to claim 16, further comprising:
a step to acquire an image based on the megavoltage treatment radiation.
